(19) United States
(12) Patent Application Publication
(10) Pub. No.: US 2006/0052368 A1
(43) Pub. Date: Mar. 9, 2006

(54) Aryl-substituted diazabicycloalkanes as nicotinic acetylcholine agonists

(76) Inventors: Glen Ernst, Wilmington, DE (US); Eifion Phillips, Wilmington, DE (US); Richard Schmiesing, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 10/524,482

(22) PCT Filed: Aug. 13, 2003
(86) PCT No.: PCT/SE03/01276

§ 371 Date: Feb. 14, 2005

(30) Foreign Application Priority Data
Aug. 14, 2002 (SE) 0202465-1

(51) Int. Cl. A61K 31/5513; A61K 31/498; C07D 487/04

(52) U.S. Cl. 514/221; 514/249; 540/558; 544/349

(57) ABSTRACT

Nicotinic acetylcholine receptor agonists of formula I 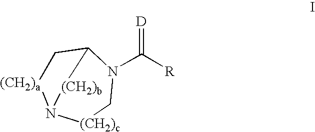 wherein a, b, c, D and R are as defined in the specification, enantiomers, pharmaceutically-acceptable salts, methods of making, pharmaceutical compositions containing and methods for using the same.

### TECHNICAL FIELD

This invention relates to diazabicycloalkane amides or pharmaceutically-acceptable salts thereof, processes for preparing them, pharmaceutical compositions containing them and their use in therapy. The invention also relates to compounds active as nicotinic acetylcholine receptors (nAChRs) agonists.

### BACKGROUND OF THE INVENTION

The use of compounds which bind nicotinic acetylcholine receptors in the treatment of a range of disorders involving reduced cholinergic function such as Alzheimer's disease, cognitive or attention disorders, anxiety, depression, smoking cessation, neuroprotection, schizophrenia, analgesia, Tourette's syndrome, and Parkinson's disease has been discussed in McDonald et al. (1995) “Nicotinic Acetylcholine Receptors: Molecular Biology, Chemistry and Pharmacology”, Chapter 5 in Annual Reports in Medicinal Chemistry, vol. 30, pp. 41-50, Academic Press Inc., San Diego, Calif.; and in Williams et al. (1994) “Neuronal Nicotinic Acetylcholine Receptors,” Drug News & Perspectives, vol. 7, pp. 205-223.

### DISCLOSURE OF THE INVENTION

The invention comprises compounds of formula I 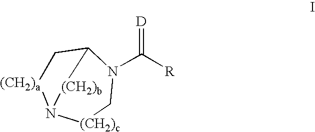
wherein:
a, b and c are each 1 or 2;
D is oxygen or sulfur, and
R is selected from moieties of formulae II, III or IV: 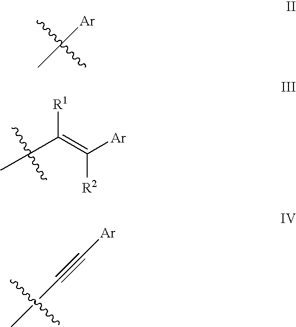
   wherein
R¹, and R² are independently selected from hydrogen, CN, CF₃, halogen, C₁₋₄alkyl, C₂₋₄alkenyl, C₂₋₄alkynyl or CO₂R³;
Ar is phenyl, or
Ar is a 5- or 6-membered aromatic heterocyclic moiety having 1, 2 or 3 heteroatoms selected from nitrogen, oxygen or sulfur where not more than one of said heteroatoms is oxygen or sulfur, or
Ar is an 8-, 9- or 10-membered fused aromatic heterocyclic moiety having 1, 2 or 3 heteroatoms selected from nitrogen, oxygen or sulfur where not more than one of said heteroatoms is oxygen or sulfur, or
Ar is an 8-, 9- or 10-membered aromatic carbocyclic ring;
said Ar phenyl, heterocyclic rings or carbocyclic having 0, 1 or more substituents independently selected from hydrogen, CN, NO₂, CF₃, halogen, C₁₋₄alkyl, C₂₋₄alkenyl, C₂₋₄alkynyl, aryl, heteroaryl, OR³, CO₂R³ or NR³R⁴; where
R³ and R⁴ are independently at each occurrence selected from hydrogen, C₁₋₄alkyl, aryl, heteroaryl, C(O)R⁵, C(O)NHR⁵, CO₂R⁵, SO₂R⁶, or
R³, R⁴ and N in combination in the substituent —NR³R⁴ is (CH₂)ⱼQ(CH₂)ₖ where Q is O, S, NR⁵, or a bond; j is 2, 3 or 4 and k is 0, 1 or 2; wherein
R⁵ at each occurrence is independently selected from hydrogen, C₁₋₄alkyl, aryl, or heteroaryl, and
R⁶ at each occurrence is independently selected from C₁₋₄alkyl, aryl, or heteroaryl.

Another embodiment of the invention comprises compounds wherein D is oxygen.

Yet another embodiment of the invention comprises compounds wherein a is 1, b is 2 and c is 1.

Still another embodiment of the invention comprises compounds wherein Ar is phenyl, or Ar is a 5- or 6-membered aromatic heterocyclic moiety having 1 or 2 heteroatoms selected from nitrogen, oxygen or sulfur where not more than one of said heteroatoms is oxygen or sulfur.

Another embodiment of the invention comprises compounds wherein Ar is a phenyl, furanyl or thiophenyl.

Particular compounds of the invention are those wherein a is 1, b is 2, c is 1, D is oxygen, R¹ and R² are hydrogen and Ar is phenyl, or Ar is a 5- or 6-membered aromatic heterocyclic moiety having 1, 2 or 3 heteroatoms selected from nitrogen, oxygen or sulfur where not more than one of said heteroatoms is oxygen or sulfur, or Ar is an 8-, 9- or 10-membered fused aromatic heterocyclic moiety having 1, 2 or 3 heteroatoms selected from nitrogen, oxygen or sulfur where not more than one of said heteroatoms is oxygen or sulfur, or Ar is an 8-, 9- or 10-membered aromatic carbocyclic ring.

Particular compounds of the invention are also those wherein Ar is selected from phenyl, 2-pyridyl, 3-pyridyl, or 4-pyridyl, 2-furanyl or 3-furanyl, 2-thienyl or 3-thienyl, benzofuran-2-yl; benzofuran-3-yl, benzo[b]thiophen-2-yl or benzo[b]thiophen-3-yl.

Particular compounds of the invention are also those wherein Ar is substituted with one or more substituents independently selected from CN, NO₂, CF₃, halogen, C₁ alkyl, C₂₋₄alkenyl, C₂₋₄alkynyl, aryl, heteroaryl, OR³, CO₂R³ or NR³R⁴.

Other particular compounds of the invention are:
(1,4-diazabicyclo[3.2.2]non-4-yl)(phenyl)methanone;
(1,4-diazabicyclo[3.2.2]non-4-yl)(3-fluorophenyl)methanone;
(1,4-diazabicyclo[3.2.2]non-4-yl)(4-fluorophenyl)methanone;
(3-chlorophenyl)(1,4-diazabicyclo[3.2.2]non-4-yl)methanone;
(4-chlorophenyl)(1,4-diazabicyclo[3.2.2]non-4-yl)methanone;
(1,4-diazabicyclo[3.2.2]non-4-yl)(3,4-dichlorophenyl)methanone;
(3-bromophenyl)(1,4-diazabicyclo[3.2.2]non-4-yl)methanone;
(4-bromophenyl)(1,4-diazabicyclo[3.2.2]non-4-yl)methanone;
(1,4-diazabicyclo[3.2.2]non-4-yl)(3-iodophenyl)methanone;
(1,4-diazabicyclo[3.2.2]non-4-yl)(4-iodophenyl)methanone;
(1,4-diazabicyclo[3.2.2]non-4-yl)(4-trifluoromethylphenyl)methanone;
(1,4-diazabicyclo[3.2.2]non-4-yl)(4-methoxyphenyl)methanone;
(1,4-diazabicyclo[3.2.2]non-4-yl)(4-trifluoromethoxyphenyl)methanone;
(5-chlorofuran-2-yl)(1,4-diazabicyclo[3.2.2]non-4-yl)methanone;
(5-bromofuran-2-yl)(1,4-diazabicyclo[3.2.2]non-4-yl)methanone;
(5-iodoofuran-2-yl)(1,4-diazabicyclo[3.2.2]non-4-yl)methanone;
(5-chlorothiophen-2-yl)(1,4-diazabicyclo[3.2.2]non-4-yl)methanone;
(5-bromothiophen-2-yl)(1,4-diazabicyclo[3.2.2]non-4-yl)methanone;
(5-iodoothiophen-2-yl)(1,4-diazabicyclo[3.2.2]non-4-yl)methanone;
(1,4-diazabicyclo[3.2.2]non-4-yl)(naphthalen-2-yl)methanone;
(1,4-diazabicyclo[3.2.2]non-4-yl)(benzofuran-2-yl)methanone;
(1,4-diazabicyclo[3.2.2]non-4-yl)(benzo[b]thiophen-2-yl)methanone;
1 (1,4-diazabicyclo[3.2.2]non-4-yl)-3-phenylpropenone;
1-(1,4-diazabicyclo[3.2.2]non-4-yl)-3-phenylpropynone;
1-(1,4-diazabicyclo[3.2.2]non-4-yl)-3-(furan-2-yl)propenone;
1-(1,4-diazabicyclo[3.2.2]non-4-yl)-3-(furan-3-yl)propenone;
1-(1,4-diazabicyclo[3.2.2]non-4-yl)-3-(thiophen-2-yl)propenone;
1-(1,4-diazabicyclo[3.2.2]non-4-yl)-3-(thiophen-3-yl)propenone;
(1,4-diazabicyclo[3.2.2]non-4-yl)(furan-2-yl)methanone;
(E)-1-(1,4-diazabicyclo[3.2.2]non-4-yl)-3-(furan-2-yl)propenone;
(E)-1-(1,4-diazabicyclo[3.2.2]non-4-yl)-3-(thiophen-2-yl)propenone;
(E)-1-(1,4-diazabicyclo[3.2.2]non-4-yl)-3-(2-methoxyphenyl)-propenone;
(E)-1-(1,4-diazabicyclo[3.2.2]non-4-yl)-3-(2-methylphenyl)propenone;
(1,4-diaza-bicyclo[3.2.2]non-4-yl)-(1H-indol-5-yl)-methanone;
(1,4-diaza-bicyclo[3.2.2]non-4-yl)-(methyl-1H-indol-2-yl)-methanone, and
(Z)-1-(1,4-diaza-bicyclo[3.2.2]non-4-yl)-2-fluoro-3-phenyl-propenone.

Most particular compounds of the invention are those of the examples herein.

Each embodiment and particular form of the invention encompass all diastereoisomers, enantiomers and pharmaceutically-acceptable derivatives and salts of compounds thereof.

Pharmaceutically-acceptable derivatives include solvates and salts. For example, the compounds of formula I can form acid addition salts with acids, such as the conventional pharmaceutically-acceptable acids, for example, maleic, hydrochloric, hydrobromic, phosphoric, acetic, fumaric, salicylic, citric, lactic, mandelic, tartaric and methanesulfonic acids.

Compounds of the invention are useful in the treatment or prophylaxis of human diseases or conditions in which activation of the α7 nicotinic receptor is beneficial as well as in the treatment or prophylaxis of psychotic disorders or intellectual impairment disorders. Examples of such conditions, diseases or disorders are Alzheimers disease, learning deficit, cognition deficit, attention deficit, memory loss, Attention Deficit Hyperactivity Disorder, Anxiety, schizophrenia, mania or manic depression, Parkinson's disease, Huntington's disease, Tourette's syndrome, neurodegenerative disorders in which there is loss of cholinergic synapse, jetlag, cessation of smoking, nicotinic addiction including that resulting from exposure to products containing nicotine, pain, for ulcerative colitis and irritable bowel disease.

As used herein, unless otherwise indicated, “C₁₋₄alkyl” includes but is not limited to methyl, ethyl, n-propyl, n-butyl, i-propyl, i-butyl, t-butyl, s-butyl moieties, whether alone or part of another group, C₁₋₄alkyl groups may be straight-chained or branched, and C₃₋₄alkyl groups include the cyclic alkyl moieties cyclopropyl and cyclobutyl. Alkyl groups referred to herein may have 1, 2 or 3 halogen substituents.

As used herein, unless otherwise indicated, “C₂₋₄alkenyl” includes but is not limited to 1-propenyl, 2-propenyl, 1-butenyl, 2-butenyl and 3-butenyl.

As used herein, unless otherwise indicated, “C₂₋₄alkynyl” includes but is not limited to ethynyl, 1-propynyl, 2-propynyl, 1-butynyl, 2-butynyl and 3-butynyl.

As used herein, unless otherwise indicated, aryl refers to a phenyl ring which may have 1, 2 or 3 substituents selected from CN, NO₂, CF₃, halogen, C₁₋₄alkyl, C₂₋₄alkenyl, C₂₋₄alkynyl, OC₁₋₄alkyl, NH₂ and CO₂C₁₋₄alkyl.

As used herein, unless otherwise indicated, heteroaryl refers to a 5- or 6-membered aromatic or heteroaromatic ring having 0, 1, 2 or 3 nitrogen atoms, 0 or 1 oxygen atom, and 0 or 1 sulfur atom, provided that the ring contains at least one nitrogen, oxygen, or sulfur atom. Heteroaryl moieties may have one or more substituents selected from CN, NO₂, CF₃, halogen, C₁₋₄alkyl, C₂₋₄alkenyl, C₂₋₄alkynyl, NH₂, CO₂H, OC₁₋₄alkyl and CO₂C₁₋₄alkyl.

As used herein, unless otherwise indicated, halogen refers to fluorine, chlorine, bromine, or iodine.

Methods of Preparation

In the reaction schemes and text that follow, D and R, unless otherwise indicated, are as defined above for formula I. The compounds of formula I may be prepared according to the methods outlined in Scheme 1.

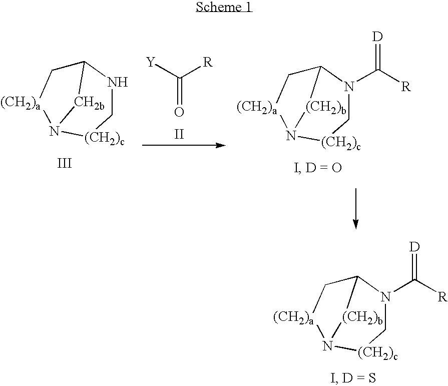

Compounds of formula I wherein D represents O may be prepared from compounds of formula III by reaction with a compound of formula II, wherein Y represents a suitable leaving group, using a suitable acylation procedure. Suitable leaving groups Y include: OH, halogen, Oalkyl, Oaryl, OCOalkyl, OCOaryl, azide. A suitable acylation procedure involves treatment of a compound of formula III with a compound of formula II at 0-120° C. in a suitable solvent. The presence of a base, or, when Y═OH, a coupling agent, may also be necessary for the reaction to occur. Suitable bases for the reaction include: 4-(N,N-dimethylamino)pyridine, pyridine, triethylamine, N,N-diisopropylethylamine. The preferred base is N,N-diisopropylethylamine. Suitable coupling agents when Y═OH include: carbodiimides, for example 1,3-dicyclohexylcarbodiimide or 1-(3-dimethylaminopropyl-3-ethylcarbodiimide hydrochloride; phosphonium reagents, for example benzotriazol-1-yloxytris(dimethylamino)phosphonium hexafluorophosphate or benzotriazol-1-yloxytripyrrolidinophosphonium hexafluorophosphate; and uronium reagents, for example O-benzotriazol-1-yl-N,N,N′,N′-tetramethyluronium tetrafluoroborate. The preferred coupling agent is O-benzotriazol-1-yl-N,N,N′,N′-tetramethyluronium tetrafluoroborate. Suitable solvents for the reaction include N,N-dimethylformamide, dimethylsulfoxide, tetrahydrofuran, or chloroform. The preferred solvent is N,N-dimethylformamide. The reaction is preferably performed at a temperature of 0-50° C., and most preferably at a temperature of 20-30° C.

Compounds of formula I in which D represents S may be prepared from compounds of formula I in which D represents O by reaction with a suitable sulfide in a suitable solvent.

The preferred sulfides are phosphorus sulfides, in particular 4-methoxyphenyl-thionophosphine sulfide dimer (“Lawesson's Reagent”), and diphosphorus pentasulfide. Suitable solvents for the reaction include aryl hydrocarbon solvents, for example toluene or xylene. The reaction is performed at a temperature of 0-200° C., and preferably at a temperature of 50-180° C.

It will be appreciated by one skilled in the art that certain optional aromatic substituents in the compounds of the invention may be introduced by employing aromatic substitution reactions, or functional group transformations to modify an existing substituent, or a combination thereof. Such reactions may be effected either prior to or immediately following the processes mentioned above, and are included as part of the process aspect of the invention. The reagents and reaction conditions for such procedures are known in the art. Specific examples of procedures which may be employed include, but are not limited to, electrophilic functionalisation of an aromatic ring, for example via nitration, halogenation, or acylation; transformation of a nitro group to an amino group, for example via reduction, such as by catalytic hydrogenation; acylation, alkylation or sulfonylation of an amino or hydroxyl group; replacement of an amino group by another functional group via conversion to an intermediate diazonium salt followed by nucleophilic or free radical substitution of the diazonium salt; or replacement of a halogen by another functional group for example via nucleophilic or catalysed substitution reactions.

Where necessary, hydroxy, amino, or other reactive groups may be protected using a protecting group as described in the standard text “Protecting groups in Organic Synthesis”, 3^{rd} Edition (1999) by Greene and Wuts.

The above described reactions, unless otherwise noted, are usually conducted at a pressure of about one to about three atmospheres, preferably at ambient pressure (about one atmosphere). Unless otherwise stated, the above described reactions are conducted-under an inert atmosphere, preferably under a nitrogen atmosphere.

The compounds of the invention and intermediates may be isolated from their reaction mixtures by standard techniques.

Acid addition salts of the compounds of formula I which may be mentioned include salts of mineral acids, for example the hydrochloride and hydrobromide salts; and salts formed with organic acids such as formate, acetate, maleate, benzoate, tartrate, and fumarate salts.

Acid addition salts of compounds of formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it nay be carried out on an ion exchange resin.

The compounds of formula I exist in tautomeric or enantiomeric forms, all of which are included within the scope of the invention. The various optical isomers may be isolated by separation of at racemic mixture of the compounds using conventional techniques, e.g. fractional crystallization, or chiral HPLC. Alternatively the individual enantiomers may be made by reaction of the appropriate optically active starting materials under reaction conditions which will not cause racemization,

### EXAMPLE 1

### (1,4-Diazabicyclo[3.2.2]non-4-yl)(phenyl)methanone

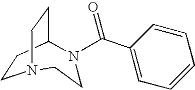

Benzoic acid (61 mg, 0.50 mmol), 1,4-diaza-bicyclo[3.2.2]nonane dihydrochloride (100 mg, 0.50 mmol), 1-hydroxybenzotriazole hydrate (68 mg, 0.50 mmol), O-(benzotriazol-1-yl)-N,N,N′,N′-tetramethyluronium tetrafluoroborate (161 mg, 0.50 mL) and diisopropylethylamine (0.35 mL, 250 mg, 2,0 mmol) in dry N,N-dimethylformamide (2 mL) wore stirred at ambient temperature for 89 h. The reaction mixture was poured into 1N sodium hydroxide solution and extracted with ethyl acetate. The ethyl acetate layer was washed with 1N NaOH (1×), water (4×), brine (1×), and dried over MgSO₄. After filtration, the solvent was removed in vacuo to yield (1,4-diaza-bicyclo[3.2.2]non-4-yl)(phenyl)methanone (13 mg, 11%) as a tan waxy solid,

MS (APCI+) 231 [M+1]⁺.

### EXAMPLE 2

### (4-Chlorophenyl)(1,4-diazabicyclo[3.2.2]non-4-yl)methanone

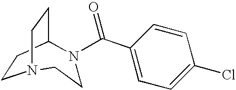

4-Chlorobenzoic acid (79 mg, 0.50 mmol), 1,4-diaza-bicyclo[3.2.2]nonane dihydrochloride (100 mg, 0.50 mmol), 1-hydroxybenzotriazole hydrate (68 mg, 0.50 mmol), O-(benzotriazol-1-yl)-N,N,N′,N′-tetramethyluronium tetrafluoroborate (161 mg, 0.50 ml) and diisopropylethylamine (0.35 mL, 250 mg, 2,0 mmol) in dry N,N-dimethylformamide (2 mL) were stirred at ambient temperature for 89 h. The reaction mixture was poured into 1N sodium hydroxide solution and extracted with ethyl acetate. The ethyl acetate layer was washed with 1N NaOH (1 x), water (4×), brine (1×), and dried over MgSO₄. After filtration, the solvent was removed in vacuo to yield (4-chlorophenyl)(1,4-diazabicyclo[3.2.2]non-4-yl)methanone (73 mg, 55%) as a tan oil.

MS (APCI+) 265/267 [M+1]+; ¹H-NMR (300 MHz, CDCl₃): δ 7.49 (2H, d), 7.40 (2H, d), 4.58-4.50 (1H, m), 3.83-3.68 (1H, m), 3.48-3.36 (1H, m), 3.02-2.75 (6H, m), 2.08-1.45 (4H, m).

### EXAMPLE 3

### (1,4-Diazabicyclo[3.2.2]non-4-yl)(4-methoxyphenyl)methanone

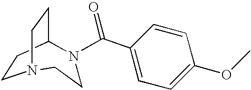

4-Methoxybenzoic acid (76 mg, 0.50 mmol), 1,4-diaza-bicyclo[3.2.2]nonane dihydrochloride (100 mg, 0.50 mmol), 1-hydroxybenzotriaole hydrate (68 mg, 0.50 mmol), O-(benzotriazol-1-yl)-N,N,N′,N′-tetramethyluronium tetrafluoroborate (161 mg, 0.50 mL) and diisopropylethylamine (0.35 mL, 250 mg, 2.0 mmol) in dry N,N-dimethylformamide (2 mL) were stirred at ambient temperature for 20 h. The reaction mixture was poured into 1N sodium hydroxide solution and extracted with ethyl acetate (2×). The ethyl acetate layers were combined and washed with water (2×). The solvent was blown off with a stream of nitrogen to yield (1,4-diazabicyclo[3.2.2]non-4-yl)(4-methoxyphenyl)methanone (13 mg, 10%) as a colorless resin.

MS (APCI+) 261 [M+1]+; ¹H-NMR (300 MHz, CDCl₃): δ 7.33 (2H, d), 6.96 (2H, d), 4.62-4.40 (1H, m), 3.80 (2H, br s), 3.78 (3H, s), 2.99-2.76 (6H, m), 2.09-1.47 (4H, m).

### EXAMPLE 4

### (1,4-Diazabicyclo[3.2.2]non-4-yl)(benzofuran-2-yl)methanone

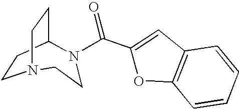

Benzofuran-2-carboxylic acid (81 mg, 0.50 mmol), 1,4-diaza-bicyclo[3.2.2]nonane dihydrochloride (100 mg, 0.50 mmol), 1-hydroxybenzotriazole hydrate (68 mg, 0.50 mmol), O-(benzotriazol-1-yl)-N,N,N′,N′-tetramethyluronium tetrafluoroborate (161 mg, 0.50 mL) and diisopropylethylamine (0.35 mL, 250 mg, 2.0 mmol) in dry N,N-dimethylformamide (2 mL) were stirred at ambient temperature for 20 h. The reaction mixture was poured into 1N sodium hydroxide solution and extracted with ethyl acetate (2×). The ethyl acetate layers wore combined and washed with water (2×). The solvent was blown off with a stream of nitrogen to yield (1,4-diazabicyclo[3.2.2]non-4-yl)(benzofuran-2-yl)methanone (46 mg, 34%) as a yellow solid.

MS (APCI+) 271 [M+1]+; ¹H-NMR (300 MHz, CDCl₃): δ 7.74 (1H, d), 7.65 (1H, d), 7.43 (1H, dd), 7.38-7.28 (2H, m), 4.59-4.38 (1H, m), 3.91-3.73 (2H, m), 3.00-2.85 (6H, m), 2.09-1.91 (2H, m), 1.83-1.64 (2H, m).

### EXAMPLE 5

### (E)-1-(1,4-Diazabicyclo[3.2.2]non-4-yl)-3-(furan-2-yl)propenone

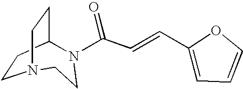

(E)-3-Furan-2-yl-acrylic acid (69 mg, 0.50 mmol), 1,4-diaza-bicyclo[3.2.2]nonane dihydrochloride (100 mg, 0.50 mmol), 1-hydroxybenzotriazole hydrate (681 mg, 0.50 mmol), O-(benzotriazol-1-yl)-N,N,N′,N′-tetramethyluronium tetrafluoroborate (161 mg, 0.50 mL) and diisopropylethylamine (0.35 mL, 250 mg, 2.0 mmol) in dry N,N-dimethylformamide (2 mL) were stirred at ambient temperature for 20 h. The reaction mixture was poured into 1N sodium hydroxide solution and extracted with ethyl acetate (2×). The ethyl acetate layers were combined and washed with water (2×). The solvent was blown off with a stream of nitrogen to yield (E)-1-(1,4-diazabicyclo[3.2.2]non-4-yl)-3-(furan-2-yl)propenone (49 mg, 40%) as a beige solid.

MS (APCI+) 247 [M+1+ ¹H-NMR (300 MHz, CDCl₃): δ 7.98-7.73 (1H, m), 7.42-7.23 (1H, m), 6.97-6.76 (2H, m), 6.63-6.53 (1H, m), 4.56-4.26 (1H, m), 3.80-3.66 (2H, m), 3.02-2.77 (6H, m), 1.97-1.53 (4H, m),

### EXAMPLE 6

### (E)-1-(1,4-Diazabicyclo[3.2.2]non-4-yl)-3-(thiophen-2-yl)propenone

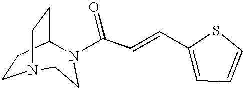

(E)-3-Thiophen-2-yl-acrylic acid (77 mg, 0.50 mmol), 1,4-diaza-bicyclo[3.2.2]nonane dihydrochloride (100 mg, 0.50 mmol), 1-hydroxybenzotriaole hydrate (68 mg, 0.50 mmol), O-(benzotriazol-1-yl)-N,N,N′,N′-tetramethyluronium tetrafluoroborate (161 mg, 0.50 mL) and O-diisopropylethylamine (0.35 mL, 250 mg, 2.0 mmol) in dry N,N-dimethylformamide (2 mL) were stirred at ambient temperature for 20 h. The reaction mixture was poured into 1N sodium hydroxide solution and extracted with ethyl acetate (2×). The ethyl acetate layers were combined and washed with water (2×). The solvent was blown off with a stream of nitrogen to yield (E)-1-(1,4-diazabicyclo[3.2.2]non-4-yl)-3-(thiophen-2-yl)propenone (62 mg, 47%) as a colorless oil.

MS (APCI+) 263 [M+1]+; ¹H-NMR (300 MHz, CDCl₃): δ 7.73-7.55 (2H, m), 7.48-7.42 (1H, m), 7.15-7.01 (1H, m), 6.96-6.76 (1H, m), 4.56-4.31 (1H, m), 3.79-3.70 (2H, m), 2.99-2.77 (6H, m), 1.97-1.54 (4H, m),

### EXAMPLE 7

### (E)-1-(1,4-Diazabicyclo[3.2.2]non-4-yl)-3-(2-methoxyphenyl)-propenone

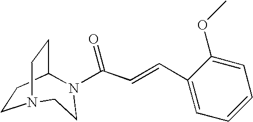

(E)-3-(2-Methoxyphenyl)acrylic acid (89 mg, 0.50 mmol), 1,4-diazabicyclo[3.2.2]nonane dihydrochloride (100 mg, 0.50 mmol), 1-hydroxybenzotriazole hydrate (68 mg, 0.50 mmol), O-(benzotriazol-1-yl)-N,N,N′,N′-tetramethyluronium tetrafluoroborate (161 mg, 0.50 mmol) and diisopropylethylamine (0.35 mL, 250 mg, 2.0 mmol) in dry N,N-dimethylformamide (2 mL) were stirred at ambient temperature for 20 h. The reaction mixture was poured into 1N sodium hydroxide solution and extracted with ethyl acetate (2×). The ethyl acetate layers were combined and washed with water (2×). The solvent was blown off with a stream of nitrogen to yield (E)-1-(1,4-diazabicyclo[3.2.2]non-4-yl)-3-(2-methoxyphenyl)propenone (74 mg, 52%) as a yellow solid.

MS (APCI+) 287 [M+1]+: ¹H-NMR (300 MHz, CDCl₃): 7.97-7.67 (2H, m), 7.41-7.30 (1H, m), 7.23-6.92 (3H, m), 4.57-4.35 (1H, m), 3.85 (3H, s), 3.81-3.72 (2H, m), 3.02-2.78 (6H, m), 1.97-1.54 (4H, m).

### EXAMPLE 8

### (E)-1-(1,4-Diazabicyclo[3.2.2]non-4-yl)-3-(2-methylphenyl)propenone

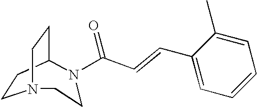

(E)-3-(2-Methylphenyl)acrylic acid (81 mg, 0.50 mmol), 1,4-diazabicyclo[3.2.2]nonane dihydrochloride (100 mg, 0.50 mmol), 1-hydroxybenzotriazole hydrate (68 mg, 0.50 mmol), O-(benzotriazol-1-yl)-N,N,N′,N′-tetramethyluronium tetrafluoroborate (161 mg, 0.50 mL) and diisopropylethylamine (0.35 mL, 250 mg, 2.0 mmol) in dry N,N-dimethylformamide (2 mL) were stirred at ambient temperature for 20 h. The reaction mixture was poured into 1N sodium hydroxide solution and extracted with ethyl acetate (2×). The ethyl acetate layers were combined and washed with water (2×). The solvent was blown off with a stream of nitrogen to yield (E)-1-(1,4-diazabicyclo[3.2.2]non-4-yl)-3-(2 methylphenyl)propenone (76 mg, 56%) as a colorless oil.

MS (APCI+) 271 [M+1]+; ¹H-NMR (300 MHz, CDCl₃): δ 7.83-7.64 (2H, m), 7.32-7.17 (3H, m), 7.16-6.96 (1H, m), 4.57-4.41 (1H, m), 3.83-3.72 (2H, m), 3.00-2.77 (6H, m), 2.37 (3H, s), 2.00-1.54 (4H, m).

### EXAMPLE 9

### (1,4-Diaza-bicyclo[3.2.2]non-4-yl)-(1H-indol-5-yl)-methanone

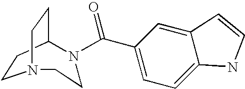

Indole-5-carboxylic acid (40 mg, 0.25 mmol), 1,4-diaza-bicyclo[3.2.2]nonane dihydrochloride (50 mg, 0,25 mmol), 1-hydroxybenzotriazole hydrate (34 mg, 0.25 mmol), O-(benzotriazol-1-yl)-N,N,N′,N′-tetramethyluronium tetrafluoroborate (81 mg, 0.25 mmol) and diisopropylethylamine (0.17 mL, 129 mg, 1.0 mmol) in dry N₁N-dimethylformamide (1.5 mL) were stirred at ambient temperature for 24 h. The reaction mixture was poured into 1N sodium hydroxide solution and extracted with ethyl acetate. The ethyl acetate layer was washed with 1N NaOH (1×), water (4×), brine (1×), and dried over Na₂SO₄. After filtration, the solvent was removed in vacuo to yield 10 mg of product. The reaction mixture was chromatographed with 100% EtOAc to 90:10 EtOAc:7N NH₃/MeOH to give (1,4-diazabicyclo[3.2.3]non-4-yl)-(1H-indol-5-yl)-methanone (5 mg, 7%) as a pale yellow oil.

MS (APCI+) 270 [M+1]+; ¹H-NMR (300 MHz, CDCl₃): δ 8.67 (1H, s), 7.68 (1H, s), 7.35 (1H, d), 7.26-7.20 (2H, m), 6.56 (1H, s), 4.81 (1H, s), 3.67-3.66 (2H, m), 3.07-2.97 (6H, m), 2.13-2.00 (2H, m), 1.77 (3H, s).

### EXAMPLE 10

### (1,4-Diaza-bicyclo[3.2.2]non-4-yl)-(naphthylene-2-yl)-methanone

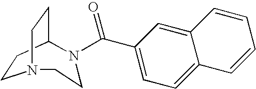

2-Napthoic acid (43 mg, 0.25 mmol), 1,4-diaza-bicyclo[3.2.2]nonane dihydrochloride (50 mg, 0.25 mmol), 1-hydroxybenzotriazole hydrate (34 mg, 0.25 mmol), O-(benzotriazol-1-yl)-N,N,N′,N′-tetramethyluronium tetrafluoroborate (81 mg, 0.25 mmol) and diisopropylethylamine (0.17 mL, 129 mg, 1,0 mmol) in dry N,N-dimethylformamide (1.5 mL) were stirred at ambient temperature for 24 h. The reaction mixture was poured into 1N sodium hydroxide solution and extracted with ethyl acetate. The ethyl acetate layer was washed with 1N NaOH (1×), water (4×), brine (1×), and dried over Na₂SO₄. After filtration, the solvent was removed in vacuo to yield 50 mg of product. The reaction mixture was chromatographed with 100% EtOAc to 90:10 EtOAc:7N NH₃/MeOH to give (1,4-diazabicyclo[3.2.2]non-4-yl)-naphthalen-2-yl-methanone (46 mg, 66%) as a colorless oil.

MS (APCI+) 281 [M+1]+, ¹H-NMR (300 MHz, CDCl₃): δ 7.89-7.84 (4H, m), 7.61-7.46 (3H, m), 4.84 (1H, s), 3.59 (1H, s), 3.15-2.94 (7H, m), 2.18 (2H, s), 1.83 (2H, s) 1.66 (1H, s).

### EXAMPLE 11

### (1,4-Diaza-bicyclo[3.2.2]non-4-yl)-(methyl-1H-indol-2-yl)-methanone

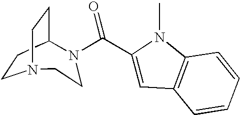

1-Methylindole-2-carboxylic acid (44 mg, 0.25 mmol), 1,4-diazabicyclo[3.2.2]nonane dihydrochloride (50 mg, 0.25 mmol), 1-hydroxybenzotriazole hydrate (34 mg, 0,25 mmol), O-(benzotriazol-1-yl)-N,N,N′,N′-tetramethyluronium tetrafluoroborate (81 mg, 0.25 mmol) and diisopropylethylamine (0.17 mL, 129 mg, 1,0 mmol) in dry N,N-dimethylformamide (1.5 mL) were stirred at ambient temperature for 24 h. The reaction mixture was poured into 1N sodium hydroxide solution and extracted with ethyl acetate. The ethyl acetate layer was washed with 1N NaOH (1×), water (4×), brine (1×), and dried over Na₂SO₄. After filtration, the solvent was removed in vacuo to yield 54 mg of product. The reaction mixture was chromatographed with 100% EtOAc to 90:10 EtOAc:7N NH₃/MeOH to give (1,4-diaza-bicyclo[3.2.2]non-4-yl)-(1-methyl-1H-indol-2-yl)-methanone (48 mg, 68%) as a colorless oil.

MS (APCI+) 284 (M+1]+; ¹H-NMR (300 MHz, CDCl₃): δ 7.62 (1H, d), 7.39-7.26 (2H, m), 7.16 (1H, dd) 6.56 (1H, s), 4.80 (1H, s), 3.86-3.77 (5H, m), 3.07-3.02 (7H, m), 2.04 (2H, s), 1.81 (2H, s) 1.66 (1H, s).

### EXAMPLE 12

### (Z)-1-(1,4-Diaza-bicyclo[3.2.2]non-4-yl)-2-fluoro-3-phenyl-propenone

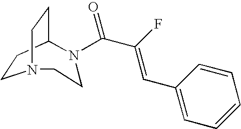

α-fluorocinnamic acid (42 mg, 0.25 mmol), 1,4-diaza-bicyclo[3.2.2]nonane dihydrochloride (50 mg, 0.25 mmol), 1-hydroxybenzotriazole hydrate (34 mg, 0.25 mmol), O-(benzotriazol-1-yl)-N,N,N′,N′-tetramethyluronium tetrafluoroborate (81 mg, 0.25 mmol) and diisopropylethylamine (0.17 mL, 129 mg, 1.0 mmol) in dry N,N-dimethylformamide (1.5 mL) wore stirred at ambient temperature for 24 h. The reaction mixture was poured into 1N sodium hydroxide solution and extracted with ethyl acetate. The ethyl acetate layer was washed with 1N NaOH (1×), water (4×), brine (1×), and dried over Na₂SO₄. After filtration, the solvent was removed in vacuo to yield 61 mg of product. The reaction mixture was chromatographed with 100% EtOAc to 90:10 EtOAc:7N NH₃/MeOH to give (Z)-1-(1,4-diaza-bicyclo[3.2.2]non-4-yl)-2-fluoro-3-phenyl-propenone (54 mg, 78%) as a colorless oil.

MS (APCI+) 275 [M+1]; ¹H-NMR (300 MHz, CDCl₃): δ 7.57 (2H, d), 7.40-7.29 (3H, m), 6.49 (1H, d), 4.62 (1H, s), 3.75 (2H, s), 3.15-2.95 (7H, m), 2.06-2.02 (2H, m), 1.79 (2H, s).

### EXAMPLE 13

### 1-(1,4-Diaza-bicyclo[3.2.2]non-4-yl)-3-phenyl-propynone

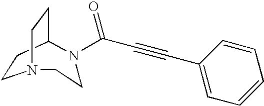

Phenylpropionic acid (37 mg, 0.25 mmol), 1,4-diaza-bicyclo[3.2.2]nonane dihydrochloride (50 mg, 0.25 mmol), 1-hydroxybenzotriazole hydrate (34 mg, 0.25 mmol), O-(benzotriazol-1-yl)-N,N,N′,N′-tetramethyluronium tetrafluoroborate (81 mg, 0.25 mmol) and diisopropylethylamine (0.17 mL, 129 mg, 1.0 mmol) in dry N,N-dimethylformamide (1.5 mL) were stirred at ambient temperature for 24 h. The reaction mixture was poured into 1N sodium hydroxide solution and extracted with ethyl acetate. The ethyl acetate layer was washed with 1N NaOH (1×), water (4×), brine (1×), and dried over Na₂SO₄. After filtration, the solvent was removed in vacuo to yield 45 mg of product. The reaction mixture was chromatographed with 100% EtOAc to 90:10 EtOAc:7N NH₃/MeOH to give 1-(1,4-diazabicyclo[3.2.2]non-4-yl)-3-phenyl-propynone (38 mg, 59%) as a colorless oil,

MS (APCI+) 255 [M+1]+; ¹H-NMR (300 MHz, CDCl₃): δ 7.61-7.51 (2H, m), 7.45-7.33 (3H, m), 4.68-4.62 (1H, m), 4.00 (1H, t), 3.86 (1H, t), 3.17-2.94 (6H, m), 2.12-1.99 (2H, m), 1.88-1.68 (3H, m).

### EXAMPLE 13

### (1,4-diazabicyclo[3.2.2]non-4-yl)(benzo[b]thiophen-2-yl)methanone dihydrochloride

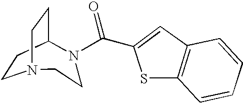

To a stirred mixture of 1,4-diazabicyclo[3.2.2]nonane dihydrochloride (100 mg, 0.51 mmol), triethylamine (0.3 mL) and a catalytic amount of N,N-dimethylaminopyridine in dry THF (2.5 mL) at ambient temperature was added a solution of benzo[b]thiophene-2-carbonyl chloride in dry THF (0.5 mL). After stirring at ambient temperature for 2 hours the mixture was partitioned between water and ethyl acetate, the organic phases recovered, washed with water and brine, then dried over sodium sulfate. The product obtained by concentration of the dried organic phases was subjected to silica gel chromatography, eluting with an ammoniated-chloroform to 5% methanol/chloroform gradient to give the title compound as a free base. The eluted material was dried to a solid. The solid was taken up in methanol, made acidic with HCl in ether (2.0 M), diluted with ether and allowed to stand. The resulting salt was collected, washed, and dried in vacuo to give the title compound as a white solid (55.0 mg). MS (ES+) 287 (MH+).

Pharmaceutical Compositions

A further aspect of the invention relates to a pharmaceutical composition for treating or preventing a condition or disorder as exemplified below arising from dysfunction of nicotinic acetylcholine receptor neurotransmission in a mammal, preferably a human, comprising an amount of a compound of formula I, an enantiomer thereof or a pharmaceutically acceptable salt thereof, effective in treating or preventing such disorder or condition and an inert pharmaceutically acceptable carrier.

For the above-mentioned uses the dosage administered will, of course, vary with the compound employed, the mode of administration and the treatment desired. However, in general, satisfactory results are obtained when the compounds of the invention are administered at a daily dosage of from about 0.1 mg to about 20 mg per kg of animal body weight, preferably given in divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carrier or diluent.

The compounds of formula I, enantiomers thereof, and pharmaceutically-acceptable salts thereof, may be used on their own or in the form of appropriate medicinal preparations for enteral or parenteral administration. According to a further aspect of the invention, there is provided a pharmaceutical composition including preferably less than 80% and more preferably less than 50% by weight of a compound of the invention in admixture with an inert pharmaceutically acceptable diluent or carrier.

Examples of diluents and carriers are:
for tablets and dragees: lactose, starch, talc, stearic acid;
capsules: tartaric acid or lactose;
for injectable solutions: water, alcohols, glycerin, vegetable oils;
for suppositories: natural or hardened oils or waxes.

There is also provided a process for the preparation of such a pharmaceutical composition, which comprises mixing the ingredients.

One aspect of the invention is the use of a compound according to the invention, an enantiomer thereof or a pharmaceutically acceptable salt thereof, in the manufacture of a medicament for the treatment or prophylaxis of one of the below mentioned diseases or conditions; and a method of treatment or prophylaxis of one of the above mentioned diseases or conditions, which comprises administering a therapeutically effective amount of a compound according to the invention, or an enantiomer thereof or a pharmaceutically acceptable salt thereof, to a patient.

Compounds to be used according to the invention are agonists of nicotinic acetylcholine receptors. While not being limited by theory, it is believed that agonists of the α₇ nAChR (nicotinic acetylcholine receptor) subtype should be useful in the treatment or prophylaxis of psychotic disorders and intellectual impairment disorders, and have advantages over compounds which are or are also agonists of the α₄ nAChR subtype. Therefore, compounds which are selective for the α₇ nAChR subtype are preferred. The use of compounds of the invention are indicated as pharmaceuticals, in particular in the treatment or prophylaxis of psychotic disorders and intellectual impairment disorders. Examples of psychotic disorders include schizophrenia, mania and manic depression, and anxiety. Examples of intellectual impairment disorders include Alzheimer's disease, learning deficit, cognition deficit, attention deficit, memory loss, and Attention Deficit Hyperactivity Disorder. The compounds of the invention may also be useful as analgesics in the treatment of pain (including chronic pain) and in the treatment or prophylaxis of Parkinson's disease, Huntington's disease, Tourette's syndrome, and neurodegenerative disorders in which there is loss of cholinergic synapses. The compounds may further be indicated for the treatment or prophylaxis of jetlag, for use in inducing the cessation of smoking, and for the treatment or prophylaxis of nicotine addiction (including that resulting from exposure to products containing nicotine).

It is also believed that compounds according to the invention are useful in the treatment and prophylaxis of ulcerative colitis.

Pharmacology

The pharmacological activity of the compounds of the invention may be measured in the tests set out below:

Test A—Assay for Affinity at α₇ nAChR Subtype

¹²⁵I-α-Bungarotoxin (BTX) Binding to Rat Hippocampal Membranes.

Rat hippocampi were homogenized in 20 volumes of cold homogenization buffer (HB: concentrations of constituents (mM): tris(hydroxymethyl)aminomethane 50; MgCl₂ 1; NaCl 120; KCl 5: pH 7.4). The homogenate was centrifuged for 5 minutes at 1000×g, the supernatant was saved and the pellet re-extracted. The pooled supernatants were centrifuged for 20 minutes at 12000×g, washed, and resuspended in HB. Membranes (30-80 μg) were incubated with 5 nM [¹²⁵I]α-BTX, 1 mg/mL BSA (bovine serum albumin), test drug, and either 2 mM CaCl₂ or 0.5 mM EGTA [ethylene glycol-bis(β-aminoethylether)] for 2 hours at 21° C., and then filtered and washed 4 times over Whatman glass fibre filters (thickness C) using a Brandel cell harvester. Pretreating the filters for 3 hours with 1% (BSA/0.01% PEI (polyethyleneimine) in water was critical for low filter blanks (0.07% of total counts per minute). Nonspecific binding was described by 100 μM (−)-nicotine, and specific binding was typically 75%.

Test B—Assay for Affinity to the α₄ nAChR Subtype

[³H]-(−)-Nicotine Binding.

Using a procedure modified from Martino-Barrows and Kellar (Mol Pharm (1987) 31:169-174), rat brain (cortex and hippocampus) was homogenized as in the [¹²⁵I]α-BTX binding assay, centrifuged for 20 minutes at 12,000×g, washed twice, and then resuspended in HB containing 100 μM diisopropyl fluorophosphate. After 20 minutes at 4° C., membranes (approximately 0.5 mg) were incubated with 3 nM [³H]-(−)-nicotine, test drug, 1 μM atropine, and either 2 mM CaCl₂ or 0.5 mM EGTA for 1 hour at 4° C., and then filtered over Whatman glass fibre filters (thickness C) (pretreated for 1 hour with 0.5% PEI) using a Brandel cell harvester. Nonspecific binding was described by 100 μM carbachol, and specific binding was typically 84%.

Binding Data Analysis for Tests A and B

IC₅₀ values and pseudo Hill coefficients (nH) were calculated using the non-linear curve fitting program ALLFIT (DeLean A, Munson P J and Rodbard D (1977) Am. J. Physiol., 235:E97-E102). Saturation curves were fitted to a one site model, using the non-linear regression program ENZFITTER (Leatherbarrow, R. J. (1987)), yielding KD values of 1.67 and 1.70 nM for the ¹²⁵I-α-BTX and [³H]-(−)-nicotine ligands respectively. Ki values were estimated using the general Cheng-Prusoff equation:
Kᵢ=[IC₅₀]/((2+([ligand]/[K_{D}])ⁿ)^{1/n}−1)
where a value of n=1 was used whenever ⁿH<1.5 and a value of n=2 was used when ⁿH>1.5. Samples were assayed in triplicate and were typically ±5%. Kᵢ values were determined using 6 or more drug concentrations. The compounds of the invention are compounds with binding affinities (Kᵢ) of less than 10 nM in either Test A or Test B, indicating that they are expected to have useful therapeutic activity.

The compounds of the invention have the advantage that they may be less toxic, be more efficacious, be longer acting, have a broader range of activity, be more potent, produce fewer side effects, are more easily absorbed or have other useful pharmacological properties.

1. A compound of formula I: 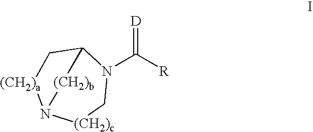
wherein:
a, b and c are each 1 or 2;
D is oxygen or sulfur, and
R is selected from moieties of formulae II, III or IV: 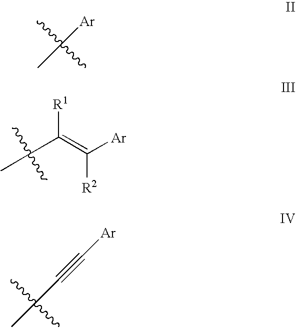
wherein
R¹, and R² are independently selected from hydrogen, CN, CF₃, halogen, C₁₋₄alkyl, C₂₋₄alkenyl, C₂₋₄alkynyl or CO₂R³;
Ar is phenyl, or
Ar is a 5- or 6-membered aromatic heterocyclic moiety having 1, 2 or 3 heteroatoms selected from nitrogen, oxygen or sulfur where not more than one of said heteroatoms is oxygen or sulfur, or
Ar is an 8-, 9- or 10-membered fused aromatic heterocyclic moiety having 1, 2 or 3 heteroatoms selected from nitrogen, oxygen or sulfur where not more than one of said heteroatoms is oxygen or sulfur, or
Ar is an 8-, 9- or 10-membered aromatic carbocyclic ring,
wherein said phenyl, heterocyclic rings or carbocyclic rings have 0, 1 or more substituents independently selected from hydrogen, CN, NO₂, CF₃, halogen, C₁₋₄alkyl, C₂₋₄alkenyl, C₂₋₄alkynyl, aryl, heteroaryl, OR³, CO₂R³ or NR³R⁴; where
R³ and R⁴ are independently at each occurrence selected from hydrogen, C₁₋₄alkyl, aryl, heteroaryl, C(O)R⁵, C(O)NHR⁵, CO₂R⁵, SO₂R⁶, or
R³, R⁴ and N in combination in the substituent —NR³R⁴ is (CH₂)ⱼQ(CH₂)ₖ where Q is O, S, NR⁵, or a bond; j is 2, 3 or 4 and k is 0, 1 or 2; wherein
R⁵ at each occurrence is independently selected from hydrogen, C₁₋₄alkyl, aryl, or heteroaryl, and
R⁶ at each occurrence is independently selected from C₁₋₄alkyl, aryl, or heteroaryl; or an enantiomer or pharmaceutically-acceptable salt thereof.

2. A compound according to claim 1, wherein D is oxygen.

3. A compound according to claim 1, wherein a is 1, b is 2 and c is 1, or an enantiomer or pharmaceutically-acceptable salt thereof.

4. A compound of claim 1, wherein
Ar is phenyl, or
Ar is a 5- or 6-membered aromatic heterocyclic moiety having 1 or 2 heteroatoms selected from nitrogen, oxygen or sulfur where not more than one of said heteroatoms is oxygen or sulfur;
or an enantiomer or pharmaceutically-acceptable salt thereof.

5. A compound according to claim 4, wherein Ar is a phenyl, furanyl or thiophenyl; or an enantiomer or pharmaceutically-acceptable salt thereof.

6. A compound according to claim 1, wherein:
a is 1;
b is 2;
c is 1;
D is oxygen;
R¹ and R² are hydrogen;
Ar is phenyl, or
Ar is a 5- or 6-membered aromatic heterocyclic moiety having 1, 2 or 3 heteroatoms selected from nitrogen, oxygen or sulfur where not more than one of said heteroatoms is oxygen or sulfur, or
Ar is an 8-, 9- or 10-membered fused aromatic heterocyclic moiety having 1, 2 or 3 heteroatoms selected from nitrogen, oxygen or sulfur where not more than one of said heteroatoms is oxygen or sulfur, or
Ar is an 8-, 9- or 10-membered aromatic carbocyclic ring;
or an enantiomer or pharmaceutically-acceptable salt thereof.

7. A compound according to claim 1, wherein:
Ar is selected from phenyl, 2-pyridyl, 3-pyridyl, or 4-pyridyl, 2-furanyl or 3-furanyl, 2-thienyl or 3-thienyl, benzofuran-2-yl; benzofuran-3-yl, benzo[b]thiophen-2-yl or benzo[b]thiophen-3-yl;
or an enantiomer or pharmaceutically-acceptable salt thereof.

8. A compound according to claim 1, wherein:
Ar is substituted with one or more substituents independently selected from CN, NO₂, CF₃, halogen, C₁₋₄alkyl, C₂₋₄alkenyl, C₂₋₄alkynyl, aryl, heteroaryl, OR³, CO₂R³ or NR³R⁴;
or an enantiomer or pharmaceutically-acceptable salt thereof.

9. A compound according to claim 1 selected from:
(1,4-diazabicyclo[3.2.2]non-4-yl)(phenyl)methanone;
(1,4-diazabicyclo[3.2.2]non-4-yl)(3-fluorophenyl)methanone;
(1,4-diazabicyclo[3.2.2]non-4-yl)(4-fluorophenyl)methanone;
(3-chlorophenyl)(1,4-diazabicyclo[3.2.2]non-4-yl)methanone;
(4-chlorophenyl)(1,4-diazabicyclo[3.2.2]non-4-yl)methanone;
(1,4-diazabicyclo[3.2.2]non-4-yl)(3,4-dichlorophenyl)methanone;
(3-bromophenyl)(1,4-diazabicyclo[3.2.2]non-4-yl)methanone;
(4-bromophenyl)(1,4-diazabicyclo[3.2.2]non-4-yl)methanone;
(1,4-diazabicyclo[3.2.2]non-4-yl)(3-iodophenyl)methanone;
(1,4-diazabicyclo[3.2.2]non-4-yl)(4-iodophenyl)methanone;
(1,4-diazabicyclo[3.2.2]non-4-yl)(4-trifluoromethylphenyl)methanone;
(1,4-diazabicyclo[3.2.2]non-4-yl)(4-methoxyphenyl)methanone;
(1,4-diazabicyclo[3.2.2]non-4-yl)(4-trifluoromethoxyphenyl)methanone;
(5-chlorofuran-2-yl)(1,4-diazabicyclo[3.2.2]non-4-yl)methanone;
(5-bromofuran-2-yl)(1,4-diazabicyclo[3.2.2]non-4-yl)methanone;
(5-iodoofuran-2-yl)(1,4-diazabicyclo[3.2.2]non-4-yl)methanone;
(5-chlorothiophen-2-yl)(1,4-diazabicyclo[3.2.2]non-4-yl)methanone;
(5-bromothiophen-2-yl)(1,4-diazabicyclo[3.2.2]non-4-yl)methanone;
(5-iodoothiophen-2-yl)(1,4-diazabicyclo[3.2.2]non-4-yl)methanone;
(1,4-diazabicyclo[3.2.2]non-4-yl)(naphthalen-2-yl)methanone;
(1,4-diazabicyclo[3.2.2]non-4-yl)(benzofuran-2-yl)methanone;
(1,4-diazabicyclo[3.2.2]non-4-yl)(benzo[b]thiophen-2-yl)methanone;
1-(1,4-diazabicyclo[3.2.2]non-4-yl)-3-phenylpropenone;
1-(1,4-diazabicyclo[3.2.2]non-4-yl)-3-phenylpropynone;
1-(1,4-diazabicyclo[3.2.2]non-4-yl)-3-(furan-2-yl)propenone;
1-(1,4-diazabicyclo[3.2.2]non-4-yl)-3-(furan-3-yl)propenone;
1-(1,4-diazabicyclo[3.2.2]non-4-yl)-3-(thiophen-2-yl)propenone;
1-(1,4-diazabicyclo[3.2.2]non-4-yl)-3-(thiophen-3-yl)propenone;
(1,4-diazabicyclo[3.2.2]non-4-yl)(furan-2-yl)methanone;
(E)-1-(1,4-diazabicyclo[3.2.2]non-4-yl)-3-(furan-2-yl)propenone;
(E)-1-(1,4-diazabicyclo[3.2.2]non-4-yl)-3-(thiophen-2-yl)propenone;
(E)-1-(1,4-diazabicyclo[3.2.2]non-4-yl)-3-(2-methoxyphenyl)-propenone;
(E)-1-(1,4-diazabicyclo[3.2.2]non-4-yl)-3-(2-methylphenyl)propenone;
(1,4-diaza-bicyclo[3.2.2]non-4-yl)-(1H-indol-5-yl)-methanone;
(1,4-diaza-bicyclo[3.2.2]non-4-yl)-(methyl-1H-indol-2-yl)-methanone, and
(Z)-1-(1,4-diaza-bicyclo[3.2.2]non-4-yl)-2-fluoro-3-phenyl-propenone,
or an enantiomer or pharmaceutically-acceptable salt thereof.

10-12. (canceled)

13. A method of treatment or prophylaxis of psychotic disorders, intellectual impairment disorders, human diseases or conditions in which activation of the α7 nicotinic receptor is beneficial, Alzheimer's disease, learning deficit, cognition deficit, attention deficit, memory loss, Lewy Body Dementia, Attention Deficit Hyperactivity Disorder, anxiety, schizophrenia, mania or manic depression, Parkinson's disease, Huntington's disease, Tourette's syndrome, neurodegenerative disorders in which there is loss of cholinergic synapse, jetlag, cessation of smoking, nicotine addiction including that resulting from exposure to products containing nicotine, pain, or ulcerative colitis which method comprises administering a therapeutically effective amount of a compound as defined in claim 1.

14. A pharmaceutical composition comprising a compound of formula I, as defined in claim 1, together with at least one pharmaceutically-acceptable excipient or diluent.
